(19) United States
(12) Patent Application Publication
(10) Pub. No.: US 2005/0240141 A1
(43) Pub. Date: Oct. 27, 2005

(54) Stent kidney curl improvements

(76) Inventors: Peter Aliski, Newton, MA (US); Vasu Nishtala, Snellville, GA (US); Benedict Shia, Needham, MA (US); Anthony Tremaglio, Charlestown, MA (US)

Correspondence Address: GANZ LAW, P.C., P O BOX 2200, HILLSBORO, OR, 97123, US

(21) Appl. No.: 10/832,889

(22) Filed: Apr. 26, 2004

(51) Int. Cl. A61M 5/00

(52) U.S. Cl. 604/8

(57) ABSTRACT

The present invention provides embodiments of medical devices that provide for fluid drainage while maintaining patient comfort. An embodiment of the present invention provides a stent comprising a body portion and a kidney curl portion, the body portion comprising a tubular member having an axial lumen therein adapted to provide fluid communication from one body location to another. The kidney curl portion comprises an elongated filament that has a three-dimensional configuration that substantially conforms to the three-dimensional randomly convoluting calyx space of a kidney. In another embodiment, the kidney curl portion comprises a plurality of petals adapted to flare outwardly from the body portion distal end when in a free state in the space of a kidney so as to prevent the migration of the kidney curl portion into the ureter.

### FIELD OF THE INVENTION

The present invention is related to ureteral stents, and more particularly, to methods and apparatus for stent kidney curl improvement.

### BACKGROUND

Tubular prostheses, commonly referred to as stents, are used in a variety of medical procedures. For example, stents are often used in connection with assisting drainage from the kidney through the ureter, from the liver through the biliary ducts, from the dorsal or ventral pancreas through the pancreatic ducts, from the gall bladder through the cystic, hepatic, or common bile ducts, and the like. A leading reason for stent deployment in ducts is to provide drainage to circumvent a blockage. Blockage of ducts in the body can be a serious and very painful affliction that can result in death if not promptly and effectively treated. Blockages can occur for a number of reasons. For example, stones and debris from such stones can pass from the kidney into the ureter, where they become entrapped. Similarly, stones and debris can pass from the gall bladder into the bile ducts, where they become trapped. Alternatively, cysts or tumors growing against the outer wall of the ducts can cause constriction of the ducts. Similarly, internal or duct-wall cysts or tumors can act to block ducts.

The main function of ureteral stents, for example, is to bypass ureteral obstruction and to provide urinary drainage from the kidney to the bladder for a period of time, typically a few days to several months. The ureteral stent is usually provided with drainage means such as a lumen for directing fluid from the renal pelvis to the bladder. Conventional stents include openings provided along the stent for communication with the lumen to aide in drainage.

Early ureteral stents were straight. As a result, after placement into the ureter, these straight stents often migrated or were expelled from the ureter as a result of peristaltic action by the ureter. Later ureteral stents, therefore, were usually designed with means of retention on one or both ends of the stent. The retention means is intended to inhibit stent migration either upward into the kidney or downward into the bladder. Retention means that have been employed are in the form of hooks, pigtails, coils, corkscrews, malecots, barbs, mushrooms, or any other practical shape that will serve the purpose.

In addition to varying lengths, ureteral stents are also made with varying diameters, e.g., from 3 French (1 mm) to 16 French (5.28 mm), and typically, 4.5 French (1.5 mm) to 8.5 French (2.8 mm), and varying degrees of hardness. Ureteral stents with smaller diameters are usually easier to insert but may not provide sufficient drainage, whereas stents with larger diameters allow for increasing drainage capacity through the ureter but may be difficult to insert.

Current urinary stents comprise a shaft commonly made of either single or dual durometer polymer material. Current shaft designs often have unique profile cross-sections and hydrophilic or anti-microbial coatings, for example. This shaft typically resides in the ureter to provide drainage of urine after ureteroscopy procedures. Stiff ureteral stents are easier to insert than are softer stents, but once inserted can lead to increased patient discomfort. Stiff stents are less likely to accommodate the dynamic urinary tract anatomy that stretches and relaxes. The relative push-and-pull of the kidney and bladder is not accommodated for, and therefore increasing patient discomfort from contact irritation of the stent within the anatomy. In addition, the lack of accommodation of the normal body movements increases the potential for the stent to migrate or dislodge from its intended location.

Anecdotally, it is believed that the softer the material, the less irritation to the ureter, and the greater the patient comfort. The problem with making the shaft extremely soft is that its lack of stiffness makes it difficult to push the stent in place. Hence for placement, a certain axial stiffness is built in which equates to a high level of radial stiffness. Stiff stents are believed to be felt by the muscle spasm of the ureter potentially causing patient discomfort. Further, the axial stiffness of current stents may not be ideal for comfort to the urinary tract anatomy without being felt by the patent.

Presently, most available stents are either made of silicone or of a harder polymer. Silicone may increase patient comfort, but because of the softness of silicone, it is more difficult to guide the stent into the ureter. Once in the ureter, the softness of the silicone increases the likelihood of migration of the stent because rigid retention means are not available.

Thus, although stents have been designed to address one or more of the above problems specifically, there are currently no devices incorporating features that can be used to bypass most of the aforementioned disadvantages. It would thus be desirable to have a stent that provides one or more of the following attributes, easy insertion or implantation, strong retention, and increase patient comfort.

### BRIEF DESCRIPTION OF THE DRAWINGS

Like reference numbers generally indicate corresponding elements in the figures.

FIG. 1 is a front partial cross-sectional view of a ureteral stent within anatomy in accordance with an embodiment of the present invention;

FIG. 2 is a perspective view of a stent body portion and kidney curl portion in accordance with the embodiment of FIG. 1;

FIG. 3 is a perspective view of the kidney curl portion in a stowed, pre-deployed configuration, in accordance with an embodiment of the present invention;

FIG. 4 is a perspective view of a stent body portion and kidney curl portion in accordance with another embodiment of the present invention; and

FIG. 5 is a front partial cross-sectional view of a ureteral stent within anatomy in accordance with the embodiment of FIG. 4.

### SUMMARY OF THE INVENTION

The present invention provides embodiments of medical devices that provide for fluid drainage while maintaining patient comfort.

An embodiment of the present invention provides a stent comprising a body portion and a kidney curl portion, the body portion comprising a tubular member, the body portion comprising a body portion distal end and a body portion proximal end, where the tubular member has an axial lumen therein adapted to provide fluid communication from the body portion distal end to the body portion proximal end. The kidney curl portion comprises an elongated filament. The kidney curl portion has a coupling end and a terminating end, where the coupling end is coupled to the body portion distal end. The kidney curl portion has a three-dimensional configuration that substantially conforms to the three-dimensional randomly convoluting calyx space of a kidney.

One advantage of embodiments of the invention is, for example, that the resilient kidney curl portion increases patient comfort by providing enhanced flexibility to accommodate the bending and stretching of the anatomy. A second advantage of embodiments of the invention is, for example, that the resilient kidney curl portion improves stent retention within the anatomy.

In one embodiment, the kidney curl portion comprises a material exhibiting shape-memory properties wherein the kidney curl portion tends to return to a predetermined configuration when deformed.

In another embodiment, the terminating end of the kidney curl further comprises a guidewire-coupling fixture adapted to removably couple with an end of a guidewire providing a means for the guidewire to push the distal end portion into anatomy.

Another embodiment of the present invention provides a stent comprising a body portion and a kidney curl portion, the body portion comprising a tubular member, the body portion comprising a body portion distal end and a body portion proximal end, where the tubular member has an axial lumen therein adapted to provide fluid communication from the body portion distal end to the body portion proximal end. The kidney curl portion comprises a plurality of petals adapted to flare outwardly from the body portion distal end when in a free state in the space of a kidney so as to prevent the migration of the kidney curl portion into the ureter.

In one embodiment, the kidney curl portion comprises a material exhibiting shape-memory properties wherein the kidney curl portion tends to return to a predetermined configuration when deformed. In another embodiment, the stent further comprises a structural member coupled to and along substantially the length of each of the petals, the structural member adapted to impart and resiliently retain a predetermined configuration to the petals when deformed.

In yet another embodiment, at least one petal further comprises a guidewire-coupling fixture adapted to removably couple with an end of a guidewire providing a means for the guidewire to push the kidney curl into anatomy.

### DETAILED DESCRIPTION

Reference will now be made to embodiments illustrated in the drawings and specific language which will be used to describe the same. It will nevertheless be understood that no limitation of the scope of the invention is thereby intended, such alterations and further modifications in the illustrated devices, as such further applications of the principles of the invention as illustrated therein as being contemplated as would normally occur to one skilled in the art to which the invention relates.

The invention relates to embodiments of medical devices (e.g., stents) for draining fluids. The invention increases patient comfort and prevents fluid retention if a stricture in a vessel develops. For simplicity and illustrative purposes, embodiments of the invention are described herein the context of draining urine from a kidney, through a ureter, and into the bladder. However, the invention is applicable to any situation that requires drainage within a body, from a body, or from one body structure to another. One such situation is, for example, biliary drainage from the gall bladder, through the biliary ducts, and to the duodenum.

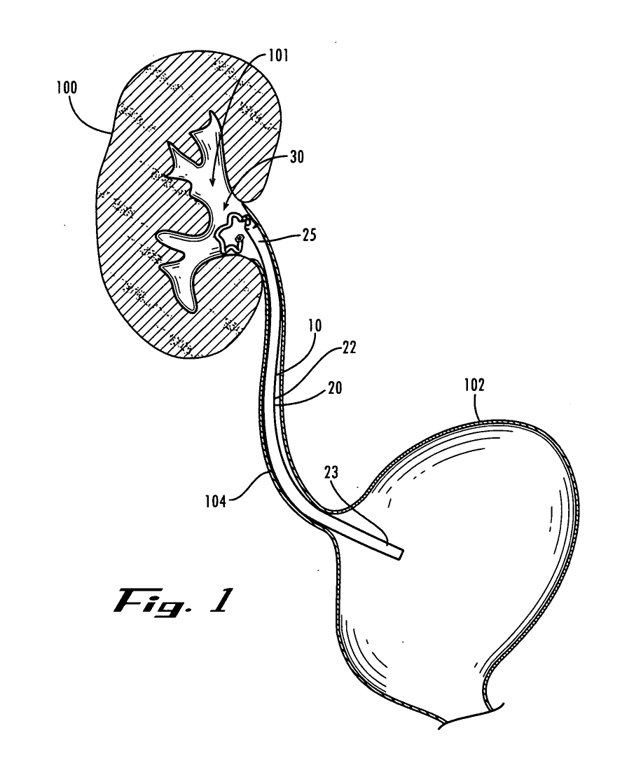

FIG. 1 is a front partial cross-sectional view of a ureteral stent 10 as deployed within anatomy in accordance with an embodiment of the present invention. The stent 10 comprises a body portion 22 and a kidney curl portion 30.

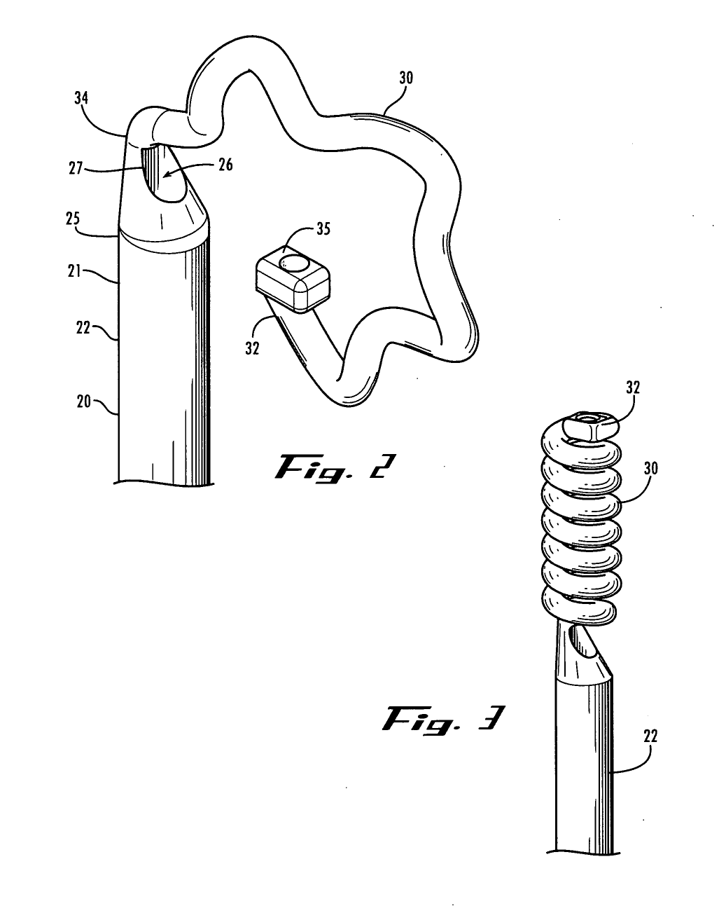

FIG. 2 is a perspective partial view of the body portion 22 and the kidney curl portion 30. The body portion 22 comprises an elongated tubular member 20 having a body portion wall 21 defining a lumen 26 therein.

The body portion 22 is adapted to be inserted into and reside substantially in the ureter 104. The kidney curl portion 30 is adapted to be inserted into and reside substantially in the calyx space 101, for example, of the kidney 100. The body portion 22 further comprises a body portion distal end 25 and a body portion proximal end 23. The body portion proximal end 23 is adapted to reside in the bladder 102. The body portion proximal end 23 of the stent 10 may have any of a variety of configurations providing a desired retaining effect or it may be entirely straight having no configuration for retention.

The lumen 26 provides a fluid path between the body portion distal end 25 and the body portion proximal end 23. The body portion distal end 25 comprises an aperture 27 in communication with the lumen 26 so as to provide a fluid path from the kidney 100 through the aperture 27, through the lumen 26 to an aperture in the proximal end 23.

The kidney curl portion 30 comprises a filament having a coupling end 34 and an opposite terminating end 32. The coupling end 34 is coupled with the body portion distal end 25. The kidney curl portion 30 is adapted to have a deployed configuration such that the kidney curl portion 30 takes on a non-planar, three-dimensional configuration that substantially conforms to the three-dimensional randomly convoluting calyx space 101 of the kidney 100. The kidney curl portion 30 is provided with a series of bends and curves to enhance retention within the kidney 100.

In one embodiment, the kidney curl portion 30 comprises a shape memory material that has a predetermined three-dimensional deployed shape-memory configuration. Upon deployment, the kidney curl portion 30 is biased by this shape memory to deploy in conformance with the predetermined shape while being sufficiently resilient to conform to the body space. In this way, the kidney curl portion 30 is caused to uncurl when deployed to stretch out and conform to the calyx space 101.

The kidney curl portion 30 comprises a material that is soft and compliant and free to deploy in a randomly convoluting space. Suitable materials for the filament includes, but is not limited to, silicone and tecoflex.

FIG. 3 is an embodiment of the kidney curl portion 30 in a pre-deployed configuration, in accordance with the present invention. The material properties of the kidney curl portion 30 is predetermined to be able to wind the kidney curl portion 30 into a helical coil having a predetermined diameter, such as, but not limited to, 4 to 6 French, for insertion through the ureter 104. Other stowed or pre-deployment configurations are anticipated suitable for a particular purpose.

In accordance with an embodiment of the present invention, the kidney curl portion 30 is stowed in a thin-walled retractable sleeve of a delivery catheter known in the art. The sleeve contains the kidney curl portion 30 in a compressed, pre-deployment position during insertion into the anatomy. Once the kidney curl portion 30 is properly positioned in the kidney 100, the sleeve is retracted and the kidney curl portion 30 expands to traverse the space within the kidney 100 and to abut and entangle with the tissues of the kidney 100.

In accordance with an embodiment of the present invention, the terminating end 32 of the kidney curl portion 30 comprises a guidewire-coupling fixture 35. The guidewire-coupling fixture 35 provides a means for accepting an end of a guidewire (not shown) so as to provide a means for pushing the kidney curl portion 30, and thus the stent 10, into the anatomy. The guidewire is inserted into the lumen 26 at the proximal end 23 and advanced to the distal end 25 where it is coupled with the guidewire-coupling fixture 35. The guidewire is coupled to the guidewire-coupling fixture 35 providing a means for the guidewire to push the kidney curl 30 through the bladder 102, in to the ureter 104, and into the kidney 100.

In one embodiment, the kidney curl portion 30 is fixed into a predetermined deployed configuration, an undulating configuration forming a generalized loop, for example. This fixation is created using any known suitable processes. In one embodiment, for example, the kidney curl portion 30 comprises a polymer material capable of retaining a predetermined shape using heat treatment techniques. For example, the kidney curl portion 30 is heated to a temperature exceeding the glass transition temperature of the polymer and shaped into a predetermined configuration.

Necessarily, the polymer is selected so that the glass transition temperature of the polymer is higher than body temperature. In this way, a “memory” of the predetermined configuration is retained by the polymer. The kidney curl portion 30, when implanted and deployed within the body, will tend to return to the predetermined configuration when stretched or otherwise deformed from the predetermined configuration. The kidney curl portion 30 can be subject to stretching or deformation, such as, during deployment, or by natural body movement.

When deployed, the kidney curl portion 30 is characterized by exhibiting a physical property that provides both longitudinal flexation allowing for the bends to stretch and recoil, as well as radial flexation allowing for bending and recovery.

The flexibility of the kidney curl portion 30 provides the stent 10 with the ability to adjust to body movements while greatly reducing the possibility of migration issues (pullout and dislodgment). Migration issues are further reduced by the length and general conformity of the kidney curl portion 30 to the folds and undulations of the interior of the kidney 100.

In an embodiment in accordance with the present invention, the kidney curl portion 30 is characterized as a region comprising a material of differing material properties than the body portion 22. The body portion 22 comprises a material having a less compliant material property (harder) transitioning to a more-compliant (softer) material for the kidney curl portion 30. In production, this transition can be accomplished by, for example, a co-extrusion process where deposition of a first material is gradually ceased and deposition of a second material is gradually increased.

The flexibility of the kidney curl portion 30 provides the ability for the stent 10 to accommodate the dynamic urinary tract anatomy that stretches and relaxes. The relative push and pull of the kidney 100 and bladder 102 is accommodated for by the very flexible fitting of the kidney curl portion 30 in the kidney 100, by resiliently bending, stretching, and recovering with the respective movements of the anatomy. The accommodation provides increased patient comfort from contact irritation of the kidney curl portion 30 within the anatomy. In addition, the accommodation reduces the potential for the kidney curl portion 30 to migrate or dislodge from its deployed location.

The stent 20 may be formed from a variety of known materials which are biocompatible and have desired physical properties to be fabricated in the form hereafter described. Examples of suitable materials are silicone, thermoplastic material, elastomers, or any material known to one skilled in the art.

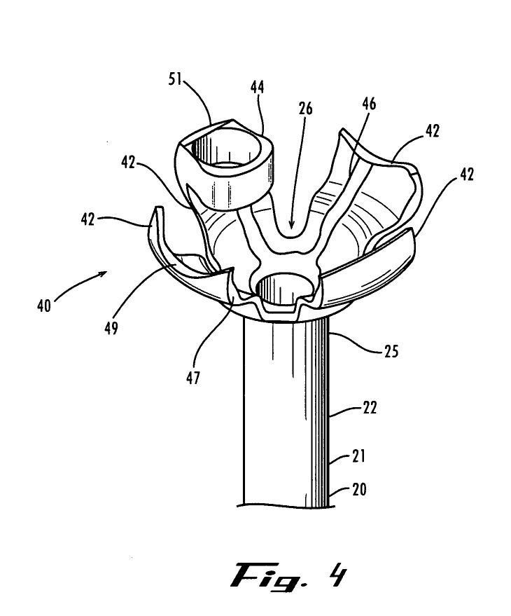

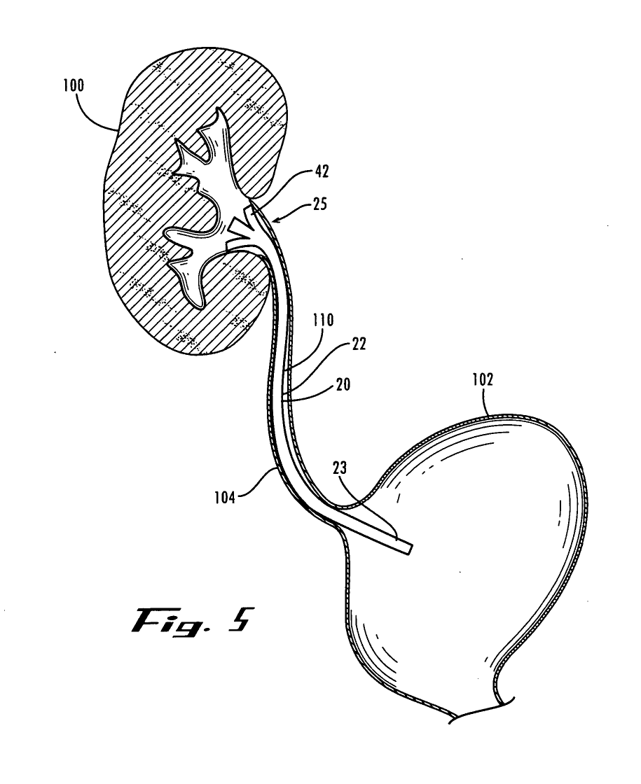

FIG. 4 is a perspective partial view of a stent 110 comprising a body portion 22 and a kidney curl portion 40, in accordance with embodiments of the present invention. FIG. 5 is a partial cut-away view of the stent 110 as provided in anatomy, shown by way of example. The body portion 22 comprises an elongated tubular member 20 having a body portion wall 21 defining a lumen 26 therein. The body portion 22 further comprises a body portion distal end 25 and a body portion proximal end 23.

The kidney curl portion 40 comprises a plurality of petals 42 adapted to flare outwardly from the body portion distal end 25 when deployed. The petals 42 comprise a predetermined length and width suitable for the particular purpose. The plurality of petals 42 provide a means for anchoring the kidney curl portion 40 in the renal pelvis, for example, and to prevent the kidney curl portion 40 from migrating into the ureter 104. Each petal 42 comprise a flexible material so as to substantially conform to the body cavity into which it is placed while sufficiently rigid to substantially retain the deployed configuration. Suitable material includes silicone, among others, as well as those materials previously discussed.

In one embodiment, the kidney curl portion 40 is fixed into a predetermined deployed configuration. This fixation is created using any known suitable process. In one embodiment, for example, the kidney curl portion 40 comprises a polymer material capable of retaining a predetermined shape using heat-treatment techniques, as discussed above.

In accordance with another embodiment of the present invention, one or more of the petals 42 is provided with a reinforcement member 46. The reinforcement member 46 is adapted to impart and/or bias a predetermined deployed shape to the petals 42 that is adapted to collapse inward for insertion and open to its free state after deployment. The reinforcement member 46, in an embodiment in accordance with the present invention, comprises a shape-memory material, such as, but not limited to, nickel-titanium alloy, such as Nitinol, that has the physical properties of super-elasticity and shape memory. The reinforcement member 46 is adapted to retain a “memory” of a predetermined configuration. The kidney curl portion 40, when implanted within the body, will tend to return to the predetermined configuration when the petals 42 are deformed from the predetermined configuration.

In an embodiment in accordance with the present invention, the petals 42 are integral with and formed from the body portion distal end 25 of the stent 110. The body portion wall 21 defining the lumen 26 is provided with a plurality of cuts 47 having a predetermined length so as to form a plurality of petals 42 each having a free end 49.

In accordance with an embodiment of the present invention, the kidney curl portion 40 is stowed in a thin-walled retractable sleeve of a delivery catheter known in the art. The sleeve contains the petals 42 in a compressed, pre-deployment position during insertion into the anatomy. Once the kidney curl portion 40 is properly positioned in the kidney 100, the sleeve is retracted and the petals 42 flare out to abut the tissues of the kidney 100.

In accordance with an embodiment of the present invention, one of the plurality of petals 42 is provided with a guidewire-coupling fixture 44. The guidewire-coupling fixture 44 provides a means for accepting an end of a guidewire (not shown) so as to provide a means for pushing the kidney curl portion 40, and thus the stent 110, into the anatomy. The guidewire is inserted into the lumen 26 at the proximal end 23 and advanced to the distal end 25 where it is coupled with the guidewire-coupling fixture 44. The guidewire is coupled to the guidewire-coupling fixture 44 providing a means for the guidewire to push the kidney curl 40 through the bladder 102, in to the ureter 104, and into the kidney 100.

After the kidney curl portion 40 has been inserted a predetermined distance into the kidney 100, the guidewire is withdrawn, enabling the kidney curl 40 to deploy. The petals 42 flare outwardly from the axis of the body portion 22 to bear against the wall of the renal pelvis and the body portion 22 against the ureter 104.

Alternatively, the ureteral stent 110 may be placed in the ureter during surgery. Other modes of inserting and/or straightening a device also known in the art.

The kidney curl portion 40 has a predetermined configuration which advantageously serves several functions. The deployed shape of the petals 42 provides means for retaining the kidney curl portion 40 in the kidney 100.

The flexibility of the kidney curl portion 40 provides the ability for the stent 110 to accommodate the dynamic urinary tract anatomy, which stretches and relaxes. The relative push and pull of the kidney 100 and bladder 102 is accommodated for by the flexible resilient property of the kidney curl portion 40 in the kidney 100, by resiliently bending, flexing, and recovering with the respective movements of the anatomy. The accommodation provides increased patient comfort from contact irritation of the kidney curl portion 40 within the anatomy. In addition, the accommodation reduces the potential for the kidney curl portion 40 to migrate or dislodge from its deployed location.

The stent 10, 110 may be formed from a variety of known materials which are biocompatible and have desired physical properties to be fabricated in the form hereafter described. Examples of suitable materials include, such as but not limited to, silicone, thermoplastic materials, elastomers, or any material known to one skilled in the art.

Confirmation that the stent 10, 110 has been placed correctly can be obtained by x-ray. If desired, radiopaque measurement markings or other suitable radiopaque indicia can be incorporated on the stent 10, 110 and are visible during x-ray examination to aid in confirming the position of the stent 10, 110.

Dimensions for the body portion 22 of the ureteral stent 10, 110 of the present invention are not critical; however, they include internal diameters of 4.5 to 8.5 French, and lengths ranging from 20 cm. to 32 cm.

The elongated member 120 according to the invention may be constructed from any of a number of materials. Those materials that are useful include, for example, materials that are able to flex but also retain their shape, to a degree, when they are perturbed. Additionally, useful materials are, for example, materials that have a resilient quality able to regain at least some of their original shape when the stent 10, 110 ceases to be perturbed and/or resist, for example, compression. One such material that combines these features is Percuflex™. Moreover, thermo-formable materials, including, for example, Percuflex™ are useful in the practice of the invention.

Identical concerns that are mentioned above with respect to stents are also applicable in the catheter and intubation arts, which include, without limitation: intravenous catheters, guiding catheters, sheaths, umbilical catheters, trocar catheters, heart catheters (including valvostomy catheters, angioplasty catheters, arthroscopy catheters, and the like), perfusion catheters, suction catheters, oxygen catheters, endoscopy catheters, endotracheal tubes, stomach tubes, feeding tubes, lavage tubes, rectal tubes, urological tubes, irrigation tubes, aneurysm shunts, stenosis dialators, trocars, and inserters, generally.

While the invention has been described in connection with specific embodiments thereof, it will be understood that it is capable of further modification, and this application is intended to cover any variations, uses, or adaptations of the invention following, the general principles of the invention and including such departures from the present disclosure as come within known or customary practice in the art to which the invention pertains and as may be applied to the essential features hereinbefore set forth, and as fall within the scope of the invention and the limits of the appended claims.

1. A stent comprising:
a body portion comprising an elongated tubular member, the body portion having a body portion proximate end and a body portion distal end; and
a kidney curl portion comprising an elongated filament, the kidney curl portion having a coupling end and a terminating end, the coupling end coupled to the body portion distal end, the tubular member having an axial lumen therein adapted to provide fluid communication from the body portion distal end to the body portion proximal end, the kidney curl portion having a three-dimensional configuration that substantially conforms to the three-dimensional randomly convoluting calyx space of a kidney.

2. The stent of claim 1, wherein the kidney curl portion comprises a resilient material so as to bend and flex to body movement and comprising a length suitable for retention within the kidney.

3. The stent of claim 1, wherein the kidney curl portion comprises a more compliant material than that comprising the body portion.

4. The stent of claim 1, wherein the kidney curl portion comprises a shape memory property having a predetermined free state configuration adapted to impart a bias on the kidney curl to a predetermined deployed state.

5. The stent of claim 1, wherein the terminating end of the kidney curl portion comprises a guidewire-coupling fixture adapted to removably couple with a guidewire.

6. A stent comprising:
a body portion comprising an elongated tubular member, the body portion having a body portion proximate end and a body portion distal end, the tubular member having an axial lumen therein adapted to provide fluid communication from the body portion distal end to the body portion proximal end; and
a kidney curl portion coupled to the body portion distal end, the kidney curl portion comprising a plurality of petals adapted to flare outwardly from the body portion distal end when in a free state.

7. The stent of claim 6, wherein the petals comprise a material exhibiting shape-memory properties wherein the petals tend to return to a predetermined configuration when deformed.

8. The stent of claim 6, further comprising a structural member coupled to and along substantially the length of each of the petals, the structural member adapted to impart and resiliently retain a predetermined configuration to the petal when deformed therefrom.

9. The stent of claim 6, wherein one of the petals further comprises:
a guidewire-coupling fixture adapted to removably couple with an end of a guidewire providing a means for the guidewire to push the kidney curl portion into anatomy.

10. A ureteral stent for maintaining drainage between a kidney and a bladder comprising:
a body portion comprising an elongated tubular member, the body portion having a body portion proximate end and a body portion distal end, the tubular member having an axial lumen therein adapted to provide fluid communication from the body portion proximate end to the body portion distal end, the body portion proximate end adapted for placement in the bladder, the body portion adapted for placement in the ureter, and the kidney portion adapted for placement in the kidney.

11. The ureteral stent of claim 10, wherein the kidney curl portion comprises a resilient material so as to bend and flex to body movement and comprising a length suitable for retention within the kidney.

12. The sent of claim 11, wherein the kidney curl portion comprises a more compliant material than that comprising the body portion.

13. The stent of claim 10, wherein the kidney curl portion comprises a shape memory property having a predetermined free state configuration adapted to impart a bias on the kidney curl to a predetermined deployed state.

14. The stent of claim 10, wherein the terminating end of the kidney curl portion comprises a guidewire-coupling fixture adapted to removably couple with a guidewire.

15. A ureteral stent for maintaining drainage between a kidney and a bladder comprising:
a body portion comprising an elongated tubular member, the body portion having a body portion proximate end and a body portion distal end, the tubular member having an axial lumen therein adapted to provide fluid communication from the body portion distal end to the body portion proximal end; and
a kidney curl portion coupled to the body portion distal end, the kidney curl portion comprising a plurality of petals adapted to flare outwardly from the body portion distal end when in a free state, the body portion proximate end adapted for placement in the bladder, the body portion adapted for placement in the ureter, and the kidney curl portion adapted for placement in the kidney and for retention in the kidney.

16. The stent of claim 15, wherein the petals comprise a material exhibiting shape-memory properties wherein the petals tend to return to a predetermined configuration when deformed.

17. The stent of claim 15, further comprising a structural member coupled to and along substantially the length of each of the petals, the structural member adapted to impart and resiliently retain a predetermined configuration to the petal when deformed therefrom.

18. The stent of claim 15 wherein one of the petals further comprises:
a guidewire-coupling fixture adapted to removably couple with an end of a guidewire providing a means for the guidewire to push the kidney curl portion into anatomy.

19. A method for placing a stent comprising:
inserting a body portion into a ureter, a distal end portion into a kidney and a proximal end portion into a bladder, the stent comprising:
a body portion comprising an elongated tubular member, the body portion having a body portion proximate end and a body portion distal end; and
a kidney curl portion comprising an elongated filament, the kidney curl portion having a coupling end and a terminating end, the coupling end coupled to the body portion distal end, the tubular member having an axial lumen therein adapted to provide fluid communication from the body portion distal end to the body portion proximal end, the kidney curl portion having a three-dimensional configuration that substantially conforms to the three-dimensional randomly convoluting calyx space of a kidney.

20. The method of claim 19, wherein the terminating end of the kidney curl portion comprises a guidewire-coupling fixture adapted to removably couple with a guidewire, the method further comprising:
inserting a guidewire into the lumen of the stent and removably coupling an end of the guidewire with the guidewire-coupling fixture; and
pushing the distal end portion into the ureter using the guidewire.
